(19) United States
(12) Patent Application Publication
(10) Pub. No.: US 2015/0224060 A1
(43) Pub. Date: Aug. 13, 2015

(54) Gastric retentive tablet compositions

(71) Applicant: David Wong, Milpitas, CA (US)

(72) Inventor: David Wong, Milpitas, CA (US)

(73) Assignee: David Wong, Milpitas, CA (US)

(21) Appl. No.: 14/701,059

(22) Filed: Apr. 30, 2015

Related U.S. Application Data
(63) Continuation-in-part of application No. 14/588,925, filed on Jan. 3, 2015.

(51) Int. Cl. A61K 9/50; A61K 31/506; A61K 9/20; A61K 31/4412

(52) U.S. Cl. CPC A61K 9/5026; A61K 31/4412; A61K 31/506; A61K 9/2086; A61K 9/2081

(57) ABSTRACT

The present invention relates to a gastric retentive tablet composition comprising: (1) coated particles consisting of a drug and an amino methacrylate copolymer, and (2) an excipient, wherein the ingredients are blended together, and then compressed into a gastric retentive tablet. Thus, the coated particles and the excipient are evenly distributed in the tablet. The excipient is selected from a group consisting of a retarding agent, a binder, a filler, a chelating agent, a diluent, a disintegrant, a lubricant, a colorant, a solubilizing agent, or a mixture thereof.

### TECHNICAL FIELD

The present application claims benefit of U.S. patent application Ser. No. 14/588925 filed Jan. 3, 2015, which is incorporated herein by reference.

The present invention relates to a gastric retentive tablet composition comprising: (1) coated particles consisting of a drug and an amino methacrylate copolymer and, (2) an excipient, wherein the ingredients are blended together, and then compressed into a gastric retentive tablet. Thus, the coated particles and the excipient are evenly distributed in the tablet. The excipient is selected from a group consisting of a retarding agent, a binder, a filler, a chelating agent, a diluent, a disintegrant, a lubricant, a colorant, a solubilizing agent, or a mixture thereof.

### BACKGROUND OF THE INVENTION

An extended-release dosage form of a medicine would, in general, improve compliance and therefore an extended-release dosage form has some distinct advantages over the conventional immediate release formulations. In addition, an extended release dosage form would lower the maximum plasma concentration, and this may result in reduced toxic effects. Some drugs are absorbed high in the upper gastrointestinal tract. A gastric retentive tablet is particularly beneficial for delivery of this type of drugs, since the dosage form would be able to keep the drug in the region of absorption for a prolonged period of time.

Monolayer tablets have been commonly used in gastric retentive dosage forms. U.S. Pat. No. 8,668,929 teaches a dosage form comprising an extended release polymer matrix comprising a dose of acetaminophen and a dose of an opioid, wherein the extended release matrix is comprised of a swellable polymer and imbibes fluid after administration to swell to a size sufficient to promote gastric retention of the matrix. U.S. Pat. No. 8,592,481 teaches a gastric retentive dosage form comprising a hydrophilic polymer that upon ingestion swells to a size sufficient to achieve retention of the dosage form in the stomach in a fed mode for a period of at least about five hours.

Member-coated monolayer tablet has also been suggested. U.S. Pat. Nos. 8,580,303 and 8,333,991 teach a dosage form comprises (a) at least one component that contains a gas generating agent and gabapentin, and (b) at least one hydrophilic membrane in the form of a sachet, which contains component (a), and wherein the hydrophilic membrane expands by inflation, floats on the aqueous phase in the stomach, and is permeable to gastric juice. U.S. Pat. No. 8,529,955, U.S. Pat. No. 8,440,232 and U.S. Pat. No. 8,475,813 suggest a dosage form comprising: a core comprising gabapentin and a pharmaceutically acceptable excipient, and a semipermeable membrane surrounding the core, the semipermeable membrane comprising a plasticizer and being permeable to a fluid in an environment of use and substantially impermeable to unsolubilized gabapentin.

Bilayer tablets have been suggested for gastric retentive dosage forms. U.S. Pat. Nos. 8,685,450 8,394,408 and U.S. Pat. No. 8,409,613 describe a drug tablet including a prolonged-release core and an immediate-release layer. While, U.S. Pat. Nos. 7,736,667, 8,329,215 and U.S. Pat. No. 8,043,630 teach a gastric retentive tablet, comprising: (a) a core comprising a first polymeric matrix with said drug dispersed therein, and (b) a shell encasing said core, wherein the shell swells upon imbibition of water to a size large enough to promote retention of the dosage form in a stomach in the fed mode. The shell may contain a drug, but less in amount compared to the core.

There are different types of medications. The limited versions of the gastric retention tablets may not meet the requirements for all medications. It would be beneficial to have other forms of gastric retentive tablets as alternatives.

### BRIEF SUMMARY OF THE INVENTION

The inventor has found a novel gastric retentive tablet composition consisting essentially of a drug, an amino methacrylate copolymer, and an excipient; wherein the amino methacrylate copolymer is an acid soluble polymer, and wherein the amino methacrylate copolymer is soluble in gastric fluid up to pH 5.0. Currently, it is marketed under the brand name of EUDRAGIT® E.

Accordingly, in one aspect, the present invention relates to a novel gastric retentive tablet comprising coated particles, methacrylic acid copolymer and a excipient, wherein the core of the coated particles essentially consists of a drug, and its coat consists of EUDRAGIT® E (amino methacrylate copolymer), and wherein the excipient is selected from a group consisting of a retarding agent, a binder, a chelating agent, a filler, a diluent, a disintegrant, a lubricant, a colorant, a solubilizing agent, or a mixture thereof. And further, the core of the coated particles does not contain an excipient, and the coat of the coated particles contains only one polymer and the polymer is EUDRAGIT® E (amino methacrylate copolymer).

In a further aspect, the present invention relates to a novel gastric retentive tablet composition, wherein the drug particle is first coated with an amino methacrylate copolymer, and then mixed with EUDRAGIT® L (methacrylic acid copolymer, Type A, NF) and other excipients, compressed into a tablet.

### DETAILED DESCRIPTION OF THE INVENTION

### Definitions

“Optional” or “optionally” means that the subsequently described circumstance may or may not occur, so that the description includes instances where the circumstance occurs and instances where it does not.

Singular forms included in the claims such as “a”, “an” and “the” include the plural reference unless expressly stated or the context clearly indicates otherwise. On the other hand, the singular form “ONE” does not include the plural reference.

“Consisting of” is a transitional phrase used in the claims of this invention. “Consisting of” excludes any element, step, or ingredient not specified in the claim.

The term “gastric retentive tablet” refers to a tablet which is able to stay in the stomach for 2-4 hours. Tablet dimensions determine if it is a gastric retentive tablet; usually a tablet with a width of about 10-11 mm shows gastric retention. (U.S. Pat. No. 8,377,453) In this invention, the width of the tablet is about 11 mm, thus, it is a gastric retentive tablet and it is also an oral pharmaceutical tablet.

The term, particle, refers to a tiny body of mass. Coated particle is a particle surrounded with a coat. A coated particle has a core (particle) and a coat. A coat is formed by dissolving a polymer in a solvent, and drying the polymer on the particle surface.

### The Invention

The present invention provides a gastric retentive tablet composition and methods for preparing such composition. There are several methods for preparing the particles of this embodiment. The first method consists of the following steps: (1) suspending drug particles in a liquid to form a drug suspension, (2) dissolving EUDRAGIT® E (amino methacrylate copolymer) in a solvent to form EUDRAGIT® E (amino methacrylate copolymer) solution, (3) adding the EUDRAGIT® E (amino methacrylate copolymer) solution into the drug suspension of Step (1), and (4) drying the mixture of Step (3) into particles. The second method consists of the following steps: (1) suspending drug particles in a fluid-bed, (2) dissolving EUDRAGIT® E (amino methacrylate copolymer) in a solvent to form EUDRAGIT® E (amino methacrylate copolymer) solution, (3) spraying EUDRAGIT® E (amino methacrylate copolymer) solution onto the drug particles of Step (1), and (4) drying particles. The third method consists of the following steps: (1) mixing drug particles in a mixer, (2) dissolving EUDRAGIT® E (amino methacrylate copolymer) in a solvent to form a EUDRAGIT® E (amino methacrylate copolymer) solution, (3) spraying the EUDRAGIT® E (amino methacrylate copolymer) solution onto the drug particles of Step (1), and then (4) drying the particles. Other particle coating methods can also be used in the invention, if applicable. The coated particles are mixed with other excipients and optionally a drug, compressed into a tablet. The tablet is optionally coated for moisture barrier, taste-masking and/or cosmetic purposes. The gastric retentive tablet may have one or more of the following characteristics: (1) the tablet width is 10.0 mm or larger, and (2) the tablet may swell in an aqueous medium.

The tablet can be formed by direct compression, granulation-compression, pellet-compression or equivalent methods. In direct compression, the particles and other excipients are well-mixed and placed in a press die, compressed to form a tablet. In granulation, a binder solution is sprayed onto a mixture of the “particles” and excipients to form granules. The granules are dried and milled to a desired particle size distribution. Then, the granules are blended with other excipients, and placed in the press-die, compressed to form a tablet. Techniques for making tablets are described in Remington's Pharmaceutical Sciences, (Arthur Osol, editor), 1553-1593 (1980). Particle-coating using fluid-bed is described in U.S. Pat. No. 8,282,957. Particle-coating using spray-drying method is described in U.S. Pat. No. 8,911,766. Particle-coating using solvent-evaporation technique is described in U.S. Pat. No. 5,223,369. Some other alternative methods can also be used for particle or particulate coating in this invention.

Accordingly, the present invention provides a gastric retentive tablet comprising a drug, EUDRAGIT® E (amino methacrylate copolymer-NF) and an excipient. In this embodiment, the tablet is optionally coated for moisture barrier, cosmetic, easy-swallowing and taste-masking purposes. And, the excipient is selected from a group consisting of a retarding agent, a binder, a filler, a diluent, a disintegrant, a lubricant, a colorant, a chelating agent, a solubilizing agent, or a mixture thereof.

In one embodiment, the gastric retentive tablet composition comprises coated particles and an excipient, wherein each coated particle consists of one core and one coat, wherein the core consists of a drug, wherein the core does not contain an excipient, wherein the coat consists of EUDRAGIT® E (amino methacrylate copolymer-NF), wherein the coated particles do not contain non-polymeric materials, wherein the coated particles do not contain a water-soluble polymer, wherein the coated particles do not contain an acid-insoluble polymer, and wherein the drug is selected from the group consisting of sorafenib tosylate, dasatinib and a mixture thereof.

In another embodiment, the gastric retentive tablet composition comprises coated particles, rosin and an excipient, wherein each coated particle consists of one core and one coat, wherein the core consists of a drug, wherein the core does not contain an excipient, wherein the coat consists of EUDRAGIT® E (amino methacrylate copolymer-NF), wherein the coated particles do not contain non-polymeric materials, wherein the coated particles do not contain rosin, wherein the coated particles do not contain a water-soluble polymer, wherein the coated particles do not contain an acid-insoluble polymer, and wherein the drug is selected from the group consisting of sorafenib tosylate, dasatinib and a mixture thereof.

In a further embodiment, the gastric retentive tablet composition comprises coated particles, EUDRAGIT® L (methacrylic acid copolymer, Type A, NF), rosin and an excipient, wherein each coated particle consists of one core and one coat, wherein the core consists of a drug, wherein the core does not contain an excipient, wherein the coat consists of EUDRAGIT® E (amino methacrylate copolymer-NF), wherein the coated particles do not contain non-polymeric materials, wherein the coated particles do not contain rosin, wherein the coated particles do not contain a water-soluble polymer, wherein the coated particles do not contain an acid-insoluble polymer, and wherein the drug is selected from the group consisting of sorafenib tosylate, dasatinib and a mixture thereof.

In one aspect, the gastric retentive tablet is a monolayer tablet, and optionally coated with a film. In another aspect, the gastric retentive tablet is a multiple-layer-tablet, wherein one layer is a placebo layer. A placebo layer contains no drug. This layer may have only one face attaching to the drug layer, e.g. a bilayer tablet. (Example 5) Alternatively, the placebo layer is the core of a tablet, wherein the placebo layer is surrounded by a drug layer completely. (Example 6) In another aspect, rosin is replaced by rosin gum in the embodiments.

As most of the oral drugs are absorbed in the small intestine, the application of the invention specially applies to anti-cancer drugs. In this invention, the preferred drug candidates are those with significant lower gastrointestinal side effects, such as diarrhea and constipation, and those absorbed mainly in the upper gastrointestinal tract. Examples of individual drugs include, but are not limited to afatinib, axitinib, bosutinib, crizotinib, dasatinib, erlotinib, fostamatinib, gefitinib, ibrutinib, imatinib, lapatinib, lenvatinib, mubritinib, nilotinib, pazopanib, pegaptanib, ponatinib, regorafenib, ruxolitinib, selumetinib, sorafenib, sunitinib, SU6656 (2,3-Dihydro-N,N-dimethyl-2-oxo-3-[(4,5,6,7-tetrahydro-1H-indol-2-yl)methylene]-1H-indole-5-sulfonamide), trametinib, tofacitinib, vandetanib, vemurafenib, vismodegib. The examples also include the corresponding varieties such as salt forms and complexes, of these molecules. Among these anti-cancer drugs, sorafenib tosylate, dasatinib and few others have similar solubility properties in certain solvents.

The amount of excipient employed will depend upon how much active agent is to be used. One excipient can perform multi-functionally. Examples of excipients include but not limited to a retarding agent, a binder, a chelating agent, a filler, a diluent, a disintegrant, a lubricant, a solubilizing agent, a colorant, a chelating agent or a mixture thereof.

Enteric polymer is a polymer soluble in an aqueous medium at pH 5.5 or above. Examples of enteric polymer include but not limited to methacrylic acid copolymer, Type A, methacrylic acid copolymer, Type B, hydroxypropyl methylcellulose acetate succinate (also known as hypromellose acetate succinate), cellulose acetate phthalate, hydroxypropyl methyl cellulose phthalate, polyvinyl acetate phthalate, alginic acid, and sodium alginate. The preferred enteric polymer is methacrylic acid copolymer, Type A, NF, marketed under the brand name of EUDRAGIT® L.

Amino methacrylate copolymer, in this invention, is a cationic copolymer based on dimethylaminoethyl methacrylate, butyl methacrylate, and methyl methacrylate. The preferred amino methacrylate copolymer is soluble in gastric fluid up to pH 5.0, while it is swellable and permeable above pH 5.0. Its chemical name is poly(butyl methacrylate-co-(2-demethylaminoeethyl) methacrylate-co-methyl methacrylate) 1:2:1. Its USP/NF name is amino methacrylate copolymer-NF. Currently, it is marketed under the brand name of EUDRAGIT® E.

Rosin is a mixture of eight closely related rosin acids characterized by three fused six-carbon rings, double bonds that vary in number and location, and a single carboxylic acid group. It generally decreases cohesive strength. One of its sources is gum rosin. Currently, it is marketed by Eastman.

Retarding material is a material retarding the drug release or slowing down the matrix erosion. Examples of retarding materials include, but are not limited to, hydroxyalkyl celluloses such as hydroxypropyl cellulose, hydroxypropylmethyl cellulose (2208, 2906 and 2910) or hydroxyethyl cellulose; polyvinyl derivatives such as povidone, crospovidone or polyvinyl alcohol; polyethylene oxides; methyl cellulose; gelatin; polysaccharides such as pregelatinized starch, partially pregelatinized starch, pullulan, dextrin, sodium alginate or gum Arabic, polyethylene glycols and some water-insoluble materials. In the invention, some embodiments specify polyethylene oxide. In fact, polyethylene oxide can be replaced with any high molecular weight polymers, preferably, a water soluble and water-swellable polymer.

Binders include, but are not limited to, starches such as potato starch, wheat starch, corn starch; microcrystalline cellulose; celluloses such as hydroxypropyl cellulose, hydroxyethyl cellulose, hydroxypropyl methylcellulose, ethyl cellulose, sodium carboxy methylcellulose; natural gums like acacia, alginic acid, guar gum; liquid glucose, dextrin, povidone, syrup, polyethylene oxide, polyvinyl pyrrolidone, poly-N-vinyl amide, polyethylene glycol, gelatin, poly propylene glycol, tragacanth, combinations thereof and other materials known to one of ordinary skill in the art and mixtures thereof.

Fillers or diluents, which include, but are not limited to sugar, dextrates, dextrin, dextrose, fructose, lactitol, mannitol, sucrose, starch, lactose, xylitol, sorbitol, talc, microcrystalline cellulose, calcium carbonate, calcium phosphate dibasic or tribasic, calcium sulphate, and the like can be used.

Lubricants may be selected from, but are not limited to, those conventionally known in the art such as magnesium, aluminum or calcium or zinc stearate, polyethylene glycol, glycerol monostearate, glyceryl monosterate, glyceryl behenate, mineral oil, sodium stearyl fumarate, stearic acid, hydrogenated vegetable oils and talc.

Glidants include, but are not limited to, silicon dioxide; magnesium trisilicate, powdered cellulose, starch, talc and tribasic calcium phosphate, calcium silicate, magnesium silicate, colloidal silicon dioxide, silicon hydrogel and other materials known to one of ordinary skill in the art.

The solubilizing agents include, but are not limited to, a surfactant, such as, for example, polysorbate 80 (marketed under the brand name of TWEEN® 80) and the like, a complexing agent, such as, for example, beta-cyclodextrins and the like, a polymer, such as, for example, poloxamer 188, and the like, a co-solvent, such as, for example, methanol and the like. The solubilizing agent may also be an acid or an alkaline, if the solubility of the drug is pH dependent.

Colorants include, but are not limited to, pharmaceutical grade dyes and pigments, red ferric oxide, yellow ferric oxide, titanium dioxide, carbon black, and indigo.

Disintegrants include, but are not limited to, crospovidone, croscarmellose-sodium, sodium starch glycolate, low-substituted hydroxypropylcellulose and other materials known to one of ordinary skill in the art.

Chelating agents include, but are not limited to, alcohol, sodium benzoate, butylated hydroxytoluene, butylated hydroxyanisole and ethylenediaminetetraacetic acid.

The finished pharmaceutical dosage form of the invention can optionally have one or more coatings such as moisture-barrier film coating, sugar coating and other coatings known in the art. Coating is not considered as a matrix in this invention.

These coating layers comprises one or more excipients selected from the group comprising coating agents, plasticizers, channeling agents, opacifiers, taste-masking agents, fillers, polishing agents, coloring agents, anti-tacking (anti-sticking) agents and the like.

Coating agents (for the finished dosage form) which are useful in the coating process, include, but not limited to, polysaccharides such as maltodextrin, alkyl celluloses such as methyl or ethyl cellulose, cellulose acetate, hydroxyalkylcelluloses (e.g. hydroxypropylcellulose or hydroxypropylmethylcelluloses); polyvinylpyrrolidone, acacia, corn, sucrose, gelatin, shellac, cellulose acetate pthalate, lipids, synthetic resins, acrylic polymers, OPADRY® coating systems, polyvinyl alcohol (PVA), copolymers of vinylpyrrolidone and vinyl acetate (e.g. marketed under the brand name of PLASDONE®) and polymers based on methacrylic acid such as those marketed under the brand name of EUDRAGIT®. These may be applied from aqueous or non-aqueous systems or combinations of aqueous and non-aqueous systems as appropriate.

Additives can be included along with the film formers to obtain satisfactory films. These additives can include plasticizers such as dibutyl phthalate, triethyl citrate, polyethylene glycol (PEG) and the like, channeling agents such as surfactants, short-chain water-soluble polymers, salts and the like, anti-tacking (anti-sticking) agents such as talc, stearic acid, magnesium stearate and colloidal silicon dioxide and the like, fillers such as talc, precipitated calcium carbonate, polishing agents such as Beeswax, carnauba wax, synthetic chlorinated wax and opacifying agents such as titanium dioxide and the like. All these excipients can be used at levels well known to the persons skilled in the art.

### EXAMPLES OF INVENTION

The foregoing examples are illustrative embodiments of the invention and are merely exemplary. A person skilled in the art may make variations and modifications without deviating from the spirit and scope of the invention. All such modifications and variations are intended to be included within the scope of the invention.

### Example 1

Sorafenib tosylate particles are suspended in the chamber of a fluid-bed. EUDRAGIT® E (amino methacrylate copolymer-NF) solution is sprayed onto the particles to form coated sorafenib tosylate particles, and dried. The coated particles, 600 mg, are mixed with polyethylene oxide, 100 mg, rosin gum, 5 mg, microcrystalline cellulose 400 mg and glycerol monostearate 20 mg, and then compressed into a tablet.

### Example 2

Sorafenib tosylate particles are suspended in the chamber of a fluid-bed. EUDRAGIT® E (amino methacrylate copolymer-NF) solution is sprayed onto the particles to form coated sorafenib tosylate particles, and dried. The coated particles, 500 mg, and then mixed with another portion of sorafenib tosylate, 200 mg, EUDRAGIT® L (methacrylic acid copolymer, Type A, NF), 90 mg, polyethylene oxide, 50 mg, microcrystalline cellulose 400 mg and glycerol monostearate 20 mg; compressed into a tablet.

### Example 3

Dasatinib particles are suspended in the chamber of a fluid-bed. EUDRAGIT® E (amino methacrylate copolymer-NF) solution is sprayed onto the particles to form coated dasatinib particles, and dried. The coated particles, 500 mg, and then mixed with another portion of dasatinib, 200 mg, EUDRAGIT® L (methacrylic acid copolymer, Type A, NF), 50 mg, rosin gum, 1 mg, polyethylene oxide, 50 mg, microcrystalline cellulose 400 mg and glycerol monostearate 20 mg; compressed into a tablet.

### Example 4

Crizotinib particles are suspended in the product chamber of a fluid bed. An EUDRAGIT® E (amino methacrylate copolymer-NF) solution is sprayed onto the particles to form coated criotnib particles. The coated particles are then mixed with EUDRAGIT® L (methacrylic acid copolymer, Type A, NF), 40 mg, microcrystalline cellulose, 800 mg, hydroxypropyl methylcellulose, high viscosity grade, 100 mg, and glycerol monostearate, 20 mg, and then compressed into a tablet.

### Example 5

Dasatinib particles are suspended in the product chamber of a fluid bed. An EUDRAGIT® E (amino methacrylate copolymer-NF) solution is sprayed onto the particles to form coated dasatinib particles. The coated particles are then mixed with EUDRAGIT® L (methacrylic acid copolymer, Type A, NF), 40 mg, rosin gum 5 mg, microcrystalline cellulose, 800 mg, hydroxypropyl methylcellulose, high viscosity grade, 100 mg, and glycerol monostearate, 20 mg, and then placed in a die. A portion of microcrystalline cellulose is placed on the top of the blend in the same die. The two powders are compressed into one single bilayer tablet.

### Example 6

Dasatinib particles are suspended in the product chamber of a fluid bed. An EUDRAGIT® E (amino methacrylate copolymer-NF) solution is sprayed onto the particles to form coated dasatinib particles. The coated particles are then mixed with EUDRAGIT® L (methacrylic acid copolymer, Type A, NF), 40 mg, rosin gum 5 mg, microcrystalline cellulose, 800 mg, hydroxypropyl methylcellulose, high viscosity grade, 100 mg, and glycerol monostearate, 20 mg, to form a drug blend. Half of the drug blend is placed in a die. A small tablet containing no drug is placed in the die. The remaining drug blend is placed in the die. The mixture is compressed into a tablet-in-tablet, wherein the core of this tablet-in-tablet has no drug.

### Example 7

Sorafenib tosylate particles are suspended in the product chamber of a fluid bed. An EUDRAGIT® E (amino methacrylate copolymer-NF) solution is sprayed onto the particles to form coated sorafenib tosylate particles. The coated particles are then mixed with EUDRAGIT® L (methacrylic acid copolymer, Type A, NF), 40 mg, rosin 5 mg, microcrystalline cellulose, 800 mg, hydroxypropyl methylcellulose, high viscosity grade, 100 mg, and glycerol monostearate, 20 mg, to form a drug blend. Half of the drug blend is placed in a die. A small tablet containing no drug is placed on the top of the drug blend in the die. The remaining drug blend is placed in the die. The mixture is compressed into a tablet-in-tablet, wherein the core of this tablet-in-tablet has no drug.

### Example 8

Sorafenib particles are suspended and mixed in an aqueous solution at pH 7, EUDRAGIT® E (amino methacrylate copolymer-NF) is dissolved in a solvent, and added into the sorafenib suspension. The suspension is stirred overnight to remove the solvent. The whole system is then spray-dried to form coated sorafenib particles. The coated sorafenib particles, polyethylene oxide, sorafenib, dasatinib, rosin and magnesium stearate are blended together for 10 minutes, and compressed into a tablet, with a width of 13 mm.

### Example 9

Sorafenib and dsatinib are co-dissolved in a solvent. The mixture is then dispersed in an aqueous medium at pH 7.0 to form a suspension. An EUDRAGIT® E (amino methacrylate copolymer-NF) solution is added to the suspension, mixed well to let the organic solvent evaporate. After the particles are coated, then whole mixture is spray-dried to form coated drug particles. The coated drug particles, EUDRAGIT® L (methacrylic acid copolymer, Type A, NF), microcrystalline cellulose particles, rosin gum, polyethylene oxide, and magnesium stearate are blended together for 10 minutes, and compressed into a tablet, with a width of 11 mm.

I claim:

1. A gastric retentive tablet composition comprising coated particles and an excipient, wherein each coated particle consists of one core and one coat, wherein the core consists of a drug, wherein the core does not contain an excipient, wherein the coat consists of EUDRAGIT® E (amino methacrylate copolymer-NF), and wherein the drug is selected from the group consisting of sorafenib tosylate, dasatinib and a mixture thereof.

2. The gastric retentive tablet composition according to claim 1 being a monolayer tablet.

3. The gastric retentive tablet composition according to claim 1 being a bilayer tablet.

4. The gastric retentive tablet composition according to claim 3, wherein one side of the placebo layer attaches to the drug layer.

5. The gastric retentive tablet composition according to claim 3, wherein the placebo layer is surrounded by the drug layer completely.

6. A gastric retentive tablet composition comprising coated particles, rosin and an excipient, wherein each coated particle consists of one core and one coat, wherein the core consists of a drug, wherein the core does not contain an excipient, wherein the coat consists of EUDRAGIT® E (amino methacrylate copolymer-NF), wherein the coated particles do not contain non-polymeric materials, wherein the coated particles do not contain rosin, wherein the coated particles do not contain a water-soluble polymer, wherein the coated particles do not contain an acid-insoluble polymer, and wherein the drug is selected from the group consisting of sorafenib tosylate, dasatinib and a mixture thereof.

7. The gastric retentive tablet composition according to claim 6 being a monolayer tablet.

8. The gastric retentive tablet composition according to claim 6 being a bilayer tablet.

9. The gastric retentive tablet composition according to claim 8, wherein one side of the placebo layer attaches to the drug layer.

10. The gastric retentive tablet composition according to claim 8, wherein the placebo layer is surrounded by the drug layer completely.

11. A gastric retentive tablet composition comprising coated particles, EUDRAGIT® L (methacrylic acid copolymer, Type A, NF), rosin and an excipient, wherein each coated particle consists of one core and one coat, wherein the core consists of a drug, wherein the core does not contain an excipient, wherein the coat consists of EUDRAGIT® E (amino methacrylate copolymer-NF), wherein the coated particles do not contain non-polymeric materials, wherein the coated particles do not contain rosin, wherein the coated particles do not contain a water-soluble polymer, wherein the coated particles do not contain an acid-insoluble polymer, and wherein the drug is selected from the group consisting of sorafenib tosylate, dasatinib and a mixture thereof.

12. The gastric retentive tablet composition according to claim 11 being a monolayer tablet.

13. The gastric retentive tablet composition according to claim 11 being a bilayer tablet.

14. The gastric retentive tablet composition according to claim 13, wherein one side of the placebo layer attaches to the drug layer.

15. The gastric retentive tablet composition according to claim 13, wherein the placebo layer is surrounded by the drug layer completely.
